(19) United States
(12) Patent Application Publication
(10) Pub. No.: US 2026/0263341 A1
(43) Pub. Date: Sep. 10, 2026

(54) COLORANT COMPOSITION FOR DYEING KERATIN FIBERS AND DYE KIT CONTAINING THE SAME

(71) Applicant: L'OREAL, Paris (FR)

(72) Inventors: Jingmiao MA, Shanghai (CN); Hao ZHOU, Shanghai (CN); Yanan WU, Shanghai (CN); Zhibing LIU, Shanghai (CN)

(73) Assignee: L'OREAL, Paris (FR)

(21) Appl. No.: 19/159,519

(22) PCT Filed: Feb. 27, 2023
(86) PCT No.: PCT/CN2023/078427

§ 371 Date: Aug. 25, 2025

(51) Int. Cl. A61K 8/44; A61K 8/22; A61K 8/37; A61K 8/41; A61K 8/46; A61K 8/49; A61K 8/81; A61Q 5/06

(52) U.S. Cl. CPC A61K 8/44; A61K 8/22; A61K 8/37; A61K 8/411; A61K 8/463; A61K 8/49; A61K 8/8152; A61Q 5/065; A61K 2800/43; A61K 2800/882

(57) ABSTRACT

A colorant composition for dyeing keratin fibers, in particular hairs, comprises: (i) at least one oxidative dye chosen from oxidation bases, optionally in combination with one or more couplers; (ii) at least one hydrophilic gelling polymer chosen from anionic acrylic copolymers; (iii) from 10 wt. % to 50 wt. % of at least one liquid polar fatty substance having a polarity index value of less than 26 mN/m, relative to the total weight of the colorant composition; and (iv) a surfactant system, comprising at least one surfactant selected from the group consisting of nonionic surfactants which are different from said liquid polar fatty substance, amphoteric surfactants, and anionic surfactants.

### TECHNICAL FIELD

The present invention relates to a colorant composition for dyeing keratin fibers, in particular hairs, and to a dye kit comprising said colorant composition and a developer composition.

### BACKGROUND

Many people have for a long time sought to modify the color of their hair, and especially to dye it, for example, to mask their grey hair. The dyeing product can comprise both at least one colorant composition and at least one developer composition. For better use, the colorant composition and the developer composition may be placed respectively in a multi-compartment package, and are mixed together immediately before use.

When using hair dyeing products (home use), consumers often expect good color performance with application easiness, especially for the dyeing products in which the colorant composition and/or the developer composition are in the form of cream.

Therefore, there is need for the dyeing products with good color performance and application easiness, even for the dyeing products in which the colorant composition and/or the developer composition are in the form of cream.

### SUMMARY OF THE INVENTION

The present invention aims at providing a colorant composition for dyeing keratin fibers, which has good stability even being stored at an elevated temperature, e.g. 45° C., for a long time, e.g. 2 months, contribute to application easiness on the keratin fibers and provide good coloring performance after application, even for the colorant composition in the form of cream.

According to the first aspect, the present invention relates to a colorant composition for dyeing keratin fibers, in particular hairs, comprising
(i) at least one oxidative dye chosen from oxidation bases, optionally in combination with one or more couplers;
(ii) at least one hydrophilic gelling polymer chosen from anionic acrylic copolymers;
(iii) from 10 wt. % to 50 wt. % of at least one liquid polar fatty substance having a polarity index value of less than 26 mN/m, relative to the total weight of the colorant composition; and
(iv) a surfactant system, comprising at least one surfactant selected from the group consisting of nonionic surfactants which are different from said liquid polar fatty substance, amphoteric surfactants, anionic surfactants and mixture thereof.

According to the second aspect, the present invention relates to a dye kit, comprising (a) a colorant composition as defined above, and (b) a developer composition comprising at least one oxidizing agent.

According to the third aspect, the present invention relates to a process for dyeing keratin fibers, in particular hairs, using the dye kit as defined above, comprising mixing the colorant composition and the developer composition immediately before use, and applying the resulted mixture onto the keratin fibers.

Other subjects and characteristics, aspects and advantages of the present invention will emerge even more clearly on reading the detailed description and the examples that follow.

### DETAILED DESCRIPTION OF THE INVENTION

In that which follows and unless otherwise indicated, the limits of a range of values are included within this range, in particular in the expressions “of between” and “ranging from . . . to . . . ”.

The articles “a” and “an”, as used herein, mean one or more when applied to any feature in embodiments of the present invention described in the specification and claims. The use of “a” and “an” does not limit the meaning to a single feature unless such a limit is specifically stated. Moreover, the expression “at least one” used in the present description is equivalent to the expression “one or more”.

Throughout the present application, the expression “comprising” is to be interpreted as encompassing all specifically mentioned features as well optional, additional, unspecified ones. As used herein, the use of the term “comprising” also discloses the embodiment wherein no material features or even no features other than the specifically mentioned features are present (such as “consisting essentially of” and “consisting of”). In the case of “consisting essentially of,” any additional compositions, materials, and/or components that materially affect the basic and novel characteristics are excluded from such an embodiment, but any compositions, materials and/or components that do not materially affect the basic and novel characteristics can be included in the embodiment.

The term “about” denoting a certain value is intended to denote a range within ±5% of the value. As one example, the phrase “about 100” denotes a range of 100±5, i.e. the range from 95 to 105. Generally, when the term “about” is used, it can be expected that similar results or effects according to the disclosure can be obtained within a range of 5% of the indicated value.

The term “keratin fiber(s)”, as used herein, means hairs, eyelashes, eyebrows, or body hairs. Preferably, the keratin fiber(s) means hairs.

### Colorant Composition

The colorant composition according to the present invention may comprise at least one oxidative dye, at least one hydrophilic gelling polymer, a surfactant system, at least one liquid polar fatty substance, and at least one solvent.

### Oxidative Dye

The oxidative dye of the present invention is generally chosen from oxidation bases, optionally combined with one or more couplers.

Preferentially, the oxidative dye comprises one or more oxidation bases.

The oxidation bases may be chosen especially from p-phenylenediamines, bis(phenyl)alkylenediamines, p-aminophenols, o-aminophenols, heterocyclic bases, and the addition salts thereof, and mixtures thereof.

Among the p-phenylenediamines, examples that may be mentioned include p-phenylenediamine, p-tolylenediamine, 2-chloro-p-phenylenediamine, 2-methyl-p-phenylenediamine (CI 76042), 3-methyl-p-phenylenediamine, 2-methoxymethyl-p-phenylenediamine, 4-methyl-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-N,N-diethyl-3-methylaniline, N,N-bis(2-hydroxyethyl)-p-phenylenediamine, 4-N,N-bis(-hydroxyethyl)amino-2-methylaniline, 4-N,N-bis(-hydroxyethyl)amino-2-chloroaniline, 2-hydroxyethyl-p-phenylenediamine, 2-fluoro-p-phenylenediamine, 2-isopropyl-p-phenylenediamine, N-(-hydroxypropyl)-p-phenylenediamine, 2-hydroxymethyl-p-phenylenediamine, N,N-dimethyl-3-methyl-p-phenylenediamine, N,N-(ethyl-hydroxyethyl)-p-phenylenediamine, N-(,-dihydroxypropyl)-p-phenylenediamine, N-(4′-aminophenyl)-p-phenylenediamine, N-phenyl-p-phenylenediamine, 2-hydroxyethyloxy-p-phenylenediamine, 2-acetylaminoethyloxy-p-phenylenediamine, N-(-methoxyethyl)-p-phenylenediamine, 4-aminophenylpyrrolidine, 2-thienyl-p-phenylenediamine, 2-hydroxyethylamino-5-aminotoluene and 3-hydroxy-1-(4′-aminophenyl)pyrrolidine, and the addition salts thereof with an acid.

Among the p-phenylenediamines mentioned above, p-phenylenediamine, p-tolylenediamine, 2-isopropyl-p-phenylenediamine, 2-hydroxyethyl-p-phenylenediamine, 2-hydroxyethyloxy-p-phenylenediamine, 2,6-dimethyl-p-phenylenediamine, 2,6-diethyl-p-phenylenediamine, 2,3-dimethyl-p-phenylenediamine, N,N-bis(-hydroxyethyl)-p-phenylenediamine, 2-chloro-p-phenylenediamine and 2-acetylaminoethyloxy-p-phenylenediamine, and the addition salts thereof with an acid, are particularly preferred.

Among the bis(phenyl)alkylenediamines, examples that may be mentioned include N,N′-bis(-hydroxyethyl)-N,N′-bis(4′-aminophenyl)-1,3-diaminopropanol, N,N′-bis(-hydroxyethyl)-N,N′-bis(4′-aminophenyl)ethylenediamine, N,N′-bis(4-aminophenyl)tetramethylenediamine, N,N′-bis(-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane and the addition salts thereof.

Among the p-aminophenols, examples that may be mentioned include p-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(-hydroxyethyl-aminomethyl)phenol and 4-amino-2-fluorophenol, and the addition salts thereof with an acid.

Among the o-aminophenols, examples that may be mentioned include 2-aminophenol, 2-amino-5-methylphenol, 2-amino-6-methylphenol and 5-acetamido-2-aminophenol, and the addition salts thereof.

Among the heterocyclic bases, examples that may be mentioned include pyridine derivatives, pyrimidine derivatives and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine and 3,4-diaminopyridine, and the addition salts thereof.

Other pyridine oxidation bases that are useful in the present invention are the 3-aminopyrazolo[1,5-a]pyridine oxidation bases or the addition salts thereof described, for example, in patent application FR 2 801 308. Examples that may be mentioned include pyrazolo[1,5-a]pyrid-3-ylamine, 2-(acetylamino)pyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol and the addition salts thereof.

Among the pyrimidine derivatives that may be mentioned are the compounds described, for example, in the patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

Among the pyrazole derivatives that may be mentioned are the compounds described in patents DE 3843892 and DE 4133957 and patent applications WO 94/08969, WO 94/08970, FR-A-2 733 749 and DE 195 43 988, for instance 4,5-diamino-1-methylpyrazole, 4,5-diamino-1-(-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(-hydroxyethyl)amino-1-methylpyrazole, and the addition salts thereof. 4,5-Diamino-1-(-methoxyethyl)pyrazole may also be used.

Preferably, a 4,5-diaminopyrazole, and more preferably 4,5-diamino-1-(-hydroxyethyl)pyrazole and/or a salt thereof is used.

Pyrazole derivatives that may also be mentioned include diamino-N,N-dihydropyrazolopyrazolones and especially those described in patent application FR-A-2 886 136, such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-bis(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one or 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or one of its salts.

Heterocyclic bases that will preferentially be used include 4,5-diamino-1-(-hydroxyethyl)pyrazole and/or 2,3-diamino-6,7-dihydro-1H,5H-pyrazolo[1,2-a]pyrazol-1-one and/or a salt thereof.

The oxidative dye may also comprise one or more couplers, which may be chosen from those conventionally used for the dyeing of keratin fibers.

Among these couplers, mention may be made especially of m-phenylenediamines, m-aminophenols, m-diphenols, naphthalene-based couplers, heterocyclic couplers, and also the addition salts thereof, and mixtures thereof.

Examples that may be mentioned include 1,3-dihydroxybenzene, 1,3-dihydroxy-2-methylbenzene, 4-chloro-1,3-dihydroxybenzene, 2,4-diamino-1-(-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-p-hydroxyethylamino-3,4-methylenedioxybenzene, -naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, 6-hydroxyindoline, hydroxyethyl-3,4-methylenedioxyaniline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole and 6-methylpyrazolo[1,5-a]benzimidazole, the addition salts thereof with an acid, and mixtures thereof, such as chlorhydrate or dichlorhydrate thereof, e.g., 1-β-hydroxyethyloxy-2,4-diamino-benzene dichlorhydrate (2,4-diaminophenoxyethanol HCl).

In general, the addition salts of the oxidation bases and couplers that may be used within the context of the invention are especially chosen from the addition salts with an acid such as the hydrochlorides, hydrobromides, sulfates, citrates, succinates, tartrates, lactates, tosylates, benzenesulfonates, phosphates and acetates.

According to the present invention, the oxidation base(s) are present in an amount ranging from about 0.1 wt. % to about 15.0 wt. %, preferably from about 0.5 wt. % to about 10.0 wt. %, or from about 1.0 wt. % to about 5.0 wt. %, relative to the total weight of the colorant composition.

According to the present invention, the coupler(s), if they are present, may be present in an amount ranging from about 0.1 wt. % to about 15.0 wt. %, preferably from about 0.5 wt. % to about 10.0 wt. %, or from about 1.0 wt. % to about 5.0 wt. %, relative to the total weight of the colorant composition.

### Hydrophilic (Aqueous) Gelling Polymer

The composition of the present invention comprises at least one hydrophilic gelling polymer chosen from anionic acrylic copolymers.

According to the present invention, the term “hydrophilic gelling polymer” means a polymer that is capable of thickening an aqueous medium. Preferably, the thickening polymer has, at 1% in water or a 50/50 water/alcohol mixture by weight at 25° C., a viscosity of greater than 100 centipoise at a shear rate of 1 s⁻¹. These viscosities can be measured using in particular viscometers or rheometers having cone-plate geometry.

For the purposes of the present invention, the term “acrylic copolymer” means a polymer resulting from the copolymerization of at least two chemically different monomers, at least one of which is chosen from unsaturated carboxylic acids, preferably acrylic acid or methacrylic acid.

According to a particular embodiment of the invention, the anionic acrylic copolymer(s) are chosen from:
anionic copolymers derived from at least one unsaturated carboxylic acid and from at least one ester of an unsaturated carboxylic acid and of a monoalcohol comprising from 1 to 6 carbon atoms and preferably from 1 to 4 carbon atoms;
anionic associative acrylic copolymers, and
mixtures thereof.

For the purposes of the present invention, the term “associative polymer” means an amphiphilic polymer that is capable, in an aqueous medium, of reversibly combining with itself or with other molecules. It generally comprises, in its chemical structure, at least one hydrophilic region or group and at least one hydrophobic region or group.

The term “hydrophobic group” means a group or a polymer bearing a saturated or unsaturated and linear or branched hydrocarbon-based chain. When it denotes a hydrocarbon-based group, the hydrophobic group comprises at least 8 carbon atoms, preferably from 10 to 30 carbon atoms, in particular from 12 to 24 carbon atoms and preferentially from 16 to 22 carbon atoms. Preferentially, the hydrocarbon-based hydrophobic group originates from a monofunctional compound. By way of example, the hydrophobic group may be derived from a fatty alcohol, such as stearyl alcohol, dodecyl alcohol or decyl alcohol, or else from a polyalkylenated fatty alcohol, such as steareth-100. It may also denote a hydrocarbon-based polymer, for instance polybutadiene.

In the context of the invention, the anionic copolymer(s) derived from at least one unsaturated carboxylic acid and from at least one ester of an unsaturated carboxylic acid and of a monoalcohol comprising from 1 to 6 carbon atoms and preferably from 1 to 4 carbon atoms are different from the anionic associative acrylic copolymer(s).

The anionic copolymer(s) derived from at least one unsaturated carboxylic acid and from at least one ester of an unsaturated carboxylic acid and of a monoalcohol comprising from 1 to 6 carbon atoms and preferably from 1 to 4 carbon atoms are copolymers comprising, among their monomers, one or more unsaturated carboxylic acids, which are more particularly α,β-monoethylenically unsaturated, and one or more esters of an unsaturated carboxylic acid, which are more particularly α,β-monoethylenically unsaturated, and of a monoalcohol comprising from 1 to 6 carbon atoms and preferably from 1 to 4 carbon atoms.

More particularly, the unsaturated carboxylic acid, which is in particular α,β-monoethylenically unsaturated, is a monomer corresponding to formula (I) below:

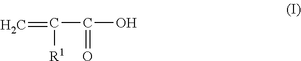

in which R¹ denotes H or CH₃ or C₂H₅, which corresponds to acrylic acid, methacrylic acid or ethacrylic acid units.

Preferably, the other monomeric ester of an unsaturated carboxylic acid and of a monoalcohol comprising from 1 to 6 carbon atoms and preferably from 1 to 4 carbon atoms is a monomer of formula (II) below:

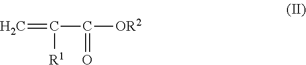

in which R¹ denotes H or CH₃ or C₂H₅ (i.e. acrylate, methacrylate or ethacrylate units) and preferably H (acrylate units) or CH₃ (methacrylate units), and R² denotes an alkyl group comprising from 1 to 6 carbon atoms and preferably from 1 to 4 carbon atoms.

As esters of an unsaturated carboxylic acid and of a fatty monoalcohol comprising from 1 to 6 carbon atoms according to formula (II), mention may be made more particularly of methyl acrylate, ethyl acrylate, propyl acrylate and butyl acrylate, and the corresponding methacrylates, methyl methacrylate, ethyl methacrylate, propyl methacrylate and butyl methacrylate.

According to one embodiment, these anionic copolymers may be crosslinked, for example, with a crosslinking agent, which is a well-known copolymerizable polyethylenic unsaturated monomer, for instance diallyl phthalate, allyl (meth)acrylate, divinylbenzene, (poly)ethylene glycol dimethacrylate or methylenebisacrylamide.

Among anionic copolymers of this type, use will more particularly be made of the polymers constituted of the following monomers:
i) of an unsaturated carboxylic acid, which is in particular α,β-monoethylenically unsaturated, corresponding to formula (I) below:

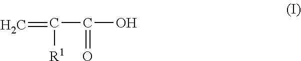

in which R¹ denotes H or CH₃ or C₂H₅, which corresponds to acrylic acid, methacrylic acid or ethacrylic acid units;
(ii) of an ester of an unsaturated carboxylic acid and of a monoalcohol comprising from 1 to 6 carbon atoms and preferably from 1 to 4 carbon atoms, of formula (II) below:

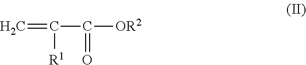

in which R¹ denotes H or CH₃ or C₂H₅ (i.e. acrylate, methacrylate or ethacrylate units) and preferably H (acrylate units) or CH₃ (methacrylate units), and R² denotes an alkyl group comprising from 1 to 6 carbon atoms and preferably from 1 to 4 carbon atoms,
(iii) and optionally a crosslinking agent, which is a well-known copolymerizable polyethylenic unsaturated monomer, such as diallyl phthalate, allyl (meth)acrylate, divinylbenzene, (poly)ethylene glycol dimethacrylate or methylenebisacrylamide.

Examples of anionic copolymers as defined above are the crosslinked copolymer of acrylic acid and of ethyl acrylate sold under the trade name Aculyn 33 by the company Rohm & Haas, which is in aqueous dispersion containing 28% by weight of active material, the methacrylic acid/ethyl acrylate crosslinked copolymer in the form of an aqueous dispersion at 30% by weight (INCI name: Acrylates Copolymer) sold under the name Carbopol Aqua SF-1 Polymer by the company Lubrizol, and the copolymer of (meth)acrylic acid and of a C₁-C₄ alkyl (meth)acrylate sold under the name Synthalen W400 by the company 3V Sigma, at 30% by weight of active material in water.

Preferably, these anionic copolymers are chosen from crosslinked copolymers of (meth)acrylic acid and of a C₁-C₄ alkyl (meth)acrylate, and better still from crosslinked copolymers of (meth)acrylic acid and of ethyl (meth)acrylate.

Among the anionic associative acrylic copolymers that may be used in the context of the invention, mention may be made of:
(1) copolymers derived from the polymerization of:
(i) (meth)acrylic acid,
(ii) a monomer of formula (III) below:

CH₂═CR′CH₂OBₙR (III)
in which R′ denotes H or CH₃, B denotes the ethyleneoxy group (—CH₂—CH₂—O—), n is zero or denotes an integer ranging from 1 to 100 (especially from 5 to 15) and R denotes a hydrocarbon-based group chosen from alkyl, arylalkyl, aryl, alkylaryl and cycloalkyl groups comprising from 8 to 30 carbon atoms, preferably from 10 to 24 carbon atoms and even more particularly from 16 to 20 carbon atoms.

A monomer of formula (III) that is more particularly preferred is a monomer in which R′ denotes H, n is equal to 10 and R denotes a stearyl (C18) group.

Such anionic associative polymers are described in patent EP-0 216 479.

Among these anionic associative polymers, the ones that are particularly preferred are polymers formed from 20% to 60% by weight of (meth)acrylic acid, from 5% to 60% by weight of C₁-C₄ alkyl (meth)acrylate, from 2% to 50% by weight of monomer of formula (III), and from 0 to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate or methylenebisacrylamide.

Among the latter polymers, preference is given most particularly to terpolymers of methacrylic acid, ethyl acrylate and polyoxyethylenated stearyl alcohol allyl ether containing 10 mol of ethylene oxide (INCI name: Steareth-10 allyl ether/acrylates copolymer), especially in 40/50/10 respective weight proportions, such as the product sold under the name Salcare SC 80 by the company Ciba;
(2) associative polymers comprising at least one hydrophilic unit of unsaturated ethylenic carboxylic acid type and at least one hydrophobic unit of (C₁₀-C₃₀) alkyl ester of unsaturated carboxylic acid type.

Preferably, these polymers are chosen from copolymers of (i) a monomer of formula (IV) below:

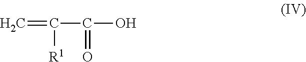

in which R¹ denotes H or CH₃ or C₂H₅, and of (ii) monomer of the following formula (V) (monomer of (C₁₀-C₃₀) alkyl ester of unsaturated carboxylic acid type):

H₂C═CR¹—COOR³ (V)
in which R¹ denotes H or CH₃ or C₂H₅ and preferably H or CH₃, R³ denotes a C₁₀-C₃₀ and preferably C₁₂-C₂₂ alkyl group.

In this polymer, the monomer (IV) constitutes the hydrophilic unit and the monomer (V) constitutes the hydrophobic unit.

(C₁₀-C₃₀) alkyl esters of unsaturated carboxylic acids comprise, for example, lauryl (meth)acrylate, stearyl (meth)acrylate, decyl (meth)acrylate, isodecyl (meth)acrylate and dodecyl (meth)acrylate.

Anionic polymers of this type are described and prepared, for example, according to patents U.S. Pat. Nos. 3,915,921 and 4,509,949.

Among the anionic associative polymers of this type that will be used more particularly are polymers formed from a monomer mixture comprising:
(i) acrylic acid,
(ii) an ester of formula (V) described above in which R¹ denotes H or CH3 and R3 denotes an alkyl group containing from 12 to 22 carbon atoms,
(iii) and optionally a crosslinking agent, which is a well-known copolymerizable polyethylenic unsaturated monomer, such as diallyl phthalate, allyl (meth)acrylate, divinylbenzene, (poly)ethylene glycol dimethacrylate or methylenebisacrylamide.

Among the anionic associative polymers of this type, use will be made more particularly of:
those constituted of 95% to 60% by weight of acrylic acid, 4% to 40% by weight of a C₁₀-C₃₀ alkyl acrylate and 0 to 6% by weight of a crosslinking polymerizable monomer, or alternatively those constituted of 98% to 96% by weight of acrylic acid, 1% to 4% by weight of a C₁₀-C₃₀ alkyl acrylate and 0.1% to 0.6% by weight of a crosslinking polymerizable monomer, such as those described previously.

Among the abovementioned polymers, the ones that are most particularly preferred are the products sold by the company Lubrizol under the trade names Pemulen TR1, Pemulen TR2, Carbopol 1382, Carbopol ETD 2020, Carbopol Ultrez 20 and Carbopol Ultrez 21 (INCI name: Acrylates/C₁₀₋₃₀ alkyl acrylate crosspolymer), and even more preferentially Pemulen TR1 and Carbopol 1382;
(3) acrylic terpolymers comprising:
(a) from 19.5% to 70% by weight of an α,β-monoethylenically unsaturated carboxylic acid containing from 3 to 5 carbon atoms,
(b) from 20% to 80% by weight of C₁-C₄ alkyl (meth)acrylates,
(c) from 0.5% to 60% by weight of a non-ionic urethane macromonomer of formula (VI) below:

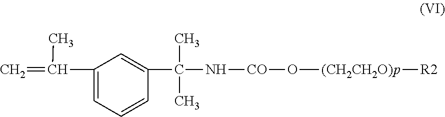

in which p ranges from 6 to 150 and R2 is chosen from linear alkyl radicals comprising from 18 to 26 and preferably from 20 to 24 carbon atoms. Preferably, the radical R2 is a behenyl radical.

Such terpolymers are described especially in patent application EP-A-0 173 109.

The α,β-monoethylenically unsaturated carboxylic acid (a) may be chosen from acrylic acid, methacrylic acid and crotonic acid. It is preferably (meth)acrylic acid. Preferentially, the monomer (a) is methacrylic acid.

The terpolymer contains a monomer (b) chosen from C₁-C₄ alkyl (meth)acrylates such as methyl (meth)acrylate, ethyl (meth)acrylate or butyl (meth)acrylate. The monomer (b) is preferably chosen from methyl acrylate and ethyl acrylate. Such terpolymers are generally in the form of an aqueous dispersion.

Use is preferentially made of a terpolymer of methacrylic acid/methyl acrylate/condensate of dimethyl m-isopropenyl benzyl isocyanate and of polyoxyethylenated (40 OE) behenyl alcohol (INCI name: Polyacrylate-3), such as the product sold in the form of an aqueous dispersion at 25% by weight, under the name Viscophobe DB 1000 by the company The Dow Chemical Company;
(4) copolymers of an α,β-monoethylenically unsaturated carboxylic acid and of an ester of an α,β-monoethylenically unsaturated carboxylic acid and of a polyoxyethylenated C₁₂-C₃₀ fatty alcohol, especially with 10 to 50 ethylene oxide units, and of an ester of an α,β-monoethylenically unsaturated carboxylic acid and of a C₁-C₄ alcohol.

Examples of such copolymers that may be mentioned include:
polymers of acrylic acid, of methyl acrylate and of 20 OE polyoxyethylenated stearyl methacrylate crosslinked with pentaerythrityl allyl ether or trimethylolpropane allyl ether (INCI name: Acrylates/steareth-20 methacrylate crosspolymer) sold under the name Aculyn 88 Polymer by the company The Dow Chemical Company,
crosslinked polymers of acrylic acid, of methyl acrylate and of 25 OE polyoxyethylenated behenyl methacrylate (INCI name: Acrylates/beheneth-25 methacrylate copolymer), such as the product sold under the name Novethix L-10 Polymer by the company Lubrizol Advanced Materials, Inc.,
polymers of acrylic acid, of methyl acrylate and of 25 OE polyoxyethylenated C12-C24 alkyl methacrylate (INCI name: Acrylates/palmeth-25 acrylate copolymer), such as the product sold under the name Synthalen W2000 L by the company 3V Group,
polymers of methacrylic acid, of ethyl methacrylate, of polyethylene glycol C16-C22 alkyl ether methacrylate containing 25 ethylene glycol units, of the ether of 2-(6,6-dimethylbicyclo[3.1.1]hept-2-en-2-yl)ethyl methacrylate and of polypropylene glycol containing 5 propylene glycol units and of polyethylene glycol containing 25 ethylene glycol units (INCI name: Polyacrylate-33), such as the product sold under the name Rheomer® 33 by the company Rhodia Novecare.
the polyoxyethylenated (20 OE) terpolymer of acrylic acid/ethyl acrylate/stearyl methacrylate (INCI name: Acrylates/steareth-20 methacrylate copolymer) sold especially under the name Aculyn 22 by the company The Dow Chemical Company,
the polyoxyethylenated (25 OE) terpolymer of acrylic acid/ethyl acrylate/behenyl methacrylate (INCI name: Acrylates/beheneth-25 methacrylate copolymer) sold especially under the name Aculyn 28 Polymer by the company The Dow Chemical Company;
(5) copolymers of (meth)acrylic acid, of crosslinked C₁-C₄ alkyl (meth)acrylate, of polyethylene glycol C₁₀-C₃₀ alkyl ether methacrylate containing 25 mol of ethylene oxide and of polyethylene glycol allyl ether containing 20 ethylene oxide units/polypropylene glycol containing 5 propylene oxide units, such as the product sold under the name Fixate® Plus Polymer by the company Lubrizol (INCI name: Polyacrylate-14).

According to one particular embodiment, the associative polymers as described above have a weight-average molecular weight of less than 500 000 and even more preferentially of less than 100 000, preferably ranging from 5000 to 80 000, which may be measured via the methods known to those skilled in the art.

Preferably, the anionic acrylic copolymers are chosen from
anionic copolymers derived from at least one unsaturated carboxylic acid and from at least one ester of an unsaturated carboxylic acid and of a monoalcohol comprising from 1 to 6 carbon atoms and preferably from 1 to 4 carbon atoms, and especially Acrylates Copolymer;
anionic associative acrylic copolymers, which are preferably chosen from copolymers of an α,β-monoethylenically unsaturated carboxylic acid, of an ester of an α,β-monoethylenically unsaturated carboxylic acid and of a polyoxyethylenated C12-C30 fatty alcohol, especially with 10 to 50 ethylene oxide units, and of an ester of an α,β-monoethylenically unsaturated carboxylic acid and of a C₁-C₄ alcohol; and especially Acrylates/steareth-20 methacrylate copolymer and Acrylates/beheneth-25 methacrylate copolymer; and
mixtures thereof.

More preferably, the anionic acrylic copolymer(s) are chosen from anionic copolymers derived from at least one unsaturated carboxylic acid and from at least one ester of an unsaturated carboxylic acid and of a monoalcohol comprising from 1 to 6 carbon atoms and preferably from 1 to 4 carbon atoms, and especially acrylates Copolymer.

According to the present invention, the hydrophilic gelling polymer chosen from anionic acrylic copolymers is present in an amount ranging from about 0.5 wt. % to about 10.0 wt. %, preferably from about 1.0 wt. % to about 5.0 wt. %, or from about 1.5 wt. % to about 3.0 wt. %, relative to the total weight of the colorant composition.

### Liquid Polar Fatty Substances

The term “fatty substance” means an organic compound insoluble in water at normal temperature (25° C.) and at atmospheric pressure (750 mmHg) (solubility below 5% and such as below 1% and further such as below 0.1%). Fatty substances have in their structure a chain of at least two siloxane groups or at least one hydrocarbon chain having at least 6 carbon atoms. Moreover, fatty substances are generally soluble in organic solvents in the same conditions of temperature and pressure, for example in chloroform, ethanol, benzene or decamethylcyclopentasiloxane.

Further, the term “liquid polar fatty substance” means the fatty substance that is liquid at normal temperature (25° C.) and at atmospheric pressure (760 mmHg, i.e. 1.013χ10⁵ Pa) and has a polarity index value of less than 26 mN/m. The term “polarity index” means the polarity or surface tension (in 10⁻³ Newton/meter), as measured by the ring method using a ring tensiometer at 20° C. against air.

The liquid polar fatty substance is, for example, chosen from fatty alcohols, esters of fatty acid, esters of fatty alcohol, oils such as vegetable, animal and synthetic non-silicone oils, silicones and mixtures thereof. Preferably, the liquid polar fatty substance is chosen from esters of fatty acid.

Examples of the liquid polar fatty substance that can be mentioned include, but not be limited to, isopropyl palmitate (25.2 mN/m), octyldodecanol (24.8 mN/m), isopropyl myristate (24.2 mN/m), ethylhexyl palmitate (23.1 mN/m), disiloxane (22.7 mN/m), isopropyl stearate (21.9 mN/m), caprylic/capric triglyceride (21.3 mN/m), isopropyl isostearate (21.2 mN/m), Jojoba Seed Oil (20.8 mN/m), Peanut Oil (20.5 mN/m), Sweet Almond Oil (20.3 mN/m), Sunflower Seed Oil (19.3 mN/m), Decyl Oleate (18.7 mN/m), Avocado Oil (18.3 mN/m), Olive Fruit Oil (16.9 mN/m), Castor Seed Oil (13.7 mN/m), Calendula officinalis Flower Oil (11.1 mN/m), Wheat Germ Oil (8.3 mN/m), and mixtures thereof.

According to the present invention, the liquid polar fatty substance is present in an amount ranging from about 10 wt. % to about 50 wt. %, preferably from about 15 wt. % to about 45 wt. %, or from about 20 wt. % to about 40 wt. %, relative to the total weight of the colorant composition.

### Surfactant System

The colorant composition of the present invention comprises a surfactant system, comprising at least one surfactant selected from the group consisting of nonionic surfactants which are different from the above liquid polar fatty substance, amphoteric surfactants, anionic surfactants and mixtures thereof.

Preferably, the surfactant system comprises at least one nonionic surfactant which are different from the above liquid polar fatty substance, at least one amphoteric surfactant, and at least one anionic surfactant.

### Amphoteric Surfactants

Useful amphoteric surfactants include betaines, alkyl sultaines, alkyl amphoacetates and alkyl amphodiacetates, alkyl amphoproprionates, and mixtures thereof. Non-limiting examples of useful amphoteric surfactants are provided below.

### (a) Betaines

Useful betaines include those of the following formulae (VIIa-VIId):

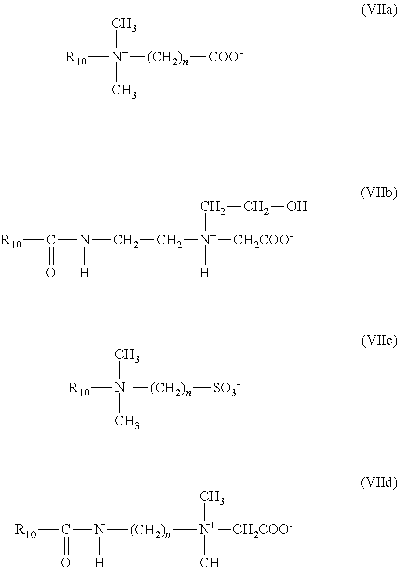

wherein R₁₀ is an alkyl group having 8-18 carbon atoms; and n is an integer from 1 to 3.

Particularly useful betaines include, for example, coco-betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and mixtures thereof. Typically, at least one betaine compound is selected from coco betaine, behenyl betaine, capryl/capramidopropyl betaine, and lauryl betaine, and mixtures thereof. Particularly preferred betaines include coco betaine and cocamidopropyl betaine.

### (b) Alkyl Sultaines

Non-limiting examples of alkyl sultaines include hydroxyl sultaines of formula (VIII)

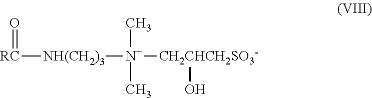

wherein R is an alkyl group having 8-18 carbon atoms. More specific examples include, but are not limited to cocamidopropyl hydroxysultaine, lauryl hydroxysultaine, and mixtures thereof.

### (c) Alkyl Amphoacetates and Alkyl Amphodiacetates

Useful alkyl amphoacetates and alkyl amphodiacetates include those of Formula (IX) and (X):

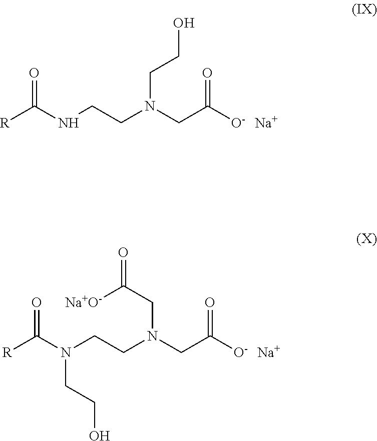

wherein R is an alkyl group having 8-18 carbon atoms. Sodium is shown as the cation in the above formulae but the cation may be an alkali metal ion such as sodium or potassium, ammonium ions, or alkanolammonium ions such as monoethanolammonium or triethanolammonium ions. A more specific, but non-limiting example, is sodium lauroamphoacetate.

### (d) Alkyl Amphopropionates

Non-limiting examples of amphopropionates include cocoamphopropionate, caprylamphopropionate, cornamphopropionate, caproamphopropionate, oleoamphopropionate, isostearoamphopropionate, stearoamphopropionate, lauroamphopropionate, salts thereof, and mixtures thereof.

According to the present invention, the amphoteric surfactant, if it is present, may be present in an amount ranging from about 0.05 wt. % to about 20 wt. %, preferably from about 0.1 wt. % to about 15 wt. %, or from about 1 wt. % to about 10 wt. %, relative to the total weight of the colorant composition.

### Anionic Surfactants

The anionic surfactants may be, for example, sulfate, sulfonate, carboxylic (or carboxylate) surfactants, or mixtures thereof. The sulfate, sulfonate, or carboxylic (or carboxylate) surfactants may, in various embodiments, comprise saturated or unsaturated hydrocarbon chains. The anionic surfactants may optionally be in salt form, or in the form of alkali metal or alkaline-earth metal, ammonium, or amino alcohol salts.

Sulfate anionic surfactants comprise at least one sulfate function. The sulfate anionic surfactants that may be used comprise at least one sulfate function (—OSO₃H or —OSO₃). They may be chosen from, by way of non-limiting example, alkyl or alkenyl sulfates, alkyl or alkenyl ether sulfates, alkylamido or alkenylamido ether sulfates, alkylaryl or alkenylaryl polyether sulfates, monoglyceride sulfates, and salts of these compounds. In various embodiments, the alkyl or alkenyl groups of these compounds comprise up to 30 carbon atoms, such as, for example from 6 to 30 carbon atoms, such as from 8 to 28, from 8 to 22 or from 8 to 18 carbon atoms, and the aryl group may optionally denote a phenyl or benzyl group. In at least some embodiments, these compounds may optionally be polyoxyalkylenated, especially polyoxyethylenated, for example comprising from 1 to 50 ethylene oxide units, such as from 2 to 10 ethylene oxide units.

In certain embodiments, sulfate anionic surfactants are chosen from alkyl or alkenyl sulfates, such as C₆-C₂₄ alkyl or alkenyl sulfates or C₁₂-C₂₀ alkyl or alkenyl sulfates, or from alkyl or alkenyl ether sulfates, optionally having from 2 to 20 ethylene oxide units, such as C₆-C₂₄ alkyl or alkenyl ether sulfates, or C₁₂-C₂₀ alkyl or alkenyl ether sulfates.

Sulfonate anionic surfactants comprise at least one sulfonate function (—SO₃H or —SO₃⁻) and may optionally also comprise one or more sulfate functions.

The sulfonate anionic surfactants that may be used comprise at least one sulfonate function (—SO₃H or —SO₃⁻). They may be chosen from the following compounds: alkylsulfonates, alkenylsulfonates, alkylamidesulfonates, alkenylamidesulfonates, alkylarylsulfonates, alkenylarylsulfonates, α-olefinsulfonates, paraffin sulfonates, alkylsulfosuccinates, alkenylsulfosuccinates, alkyl or alkenyl ether sulfosuccinates, alkylamidesulfosuccinates, alkenylamidesulfosuccinates, alkylsulfoacetates, alkenylsulfoacetates, N-acyltaurates, acylisethionates, alkylsulfolaurates, alkenylsulfolaurates, and salts of these compounds; the alkyl or alkenyl groups of these compounds comprising up to 30 carbon atoms, such as, for example, from 6 to 30 carbon atoms, such as from 8 to 28, from 8 to 22 or from 8 to 18 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

In certain embodiments, sulfonate anionic surfactants are chosen from those having up to 30 carbon atoms, such as from 6 to 30, from 8 to 28, from 8 to 22 or from 8 to 18 carbon atoms, for example alkyl or alkenyl sulfosuccinates, such as C₆-C₂₄ alkyl or alkenyl sulfosuccinates or C₈-C₁₈ alkyl or alkenyl sulfosuccinates, alkyl or alkenyl ether sulfosuccinates, such as C₆-C₂₄ alkyl or alkenyl ether sulfosuccinates or C₈-C₁₈ alkyl or alkenyl ether sulfosuccinates, or acylisethionates, such as such as C₆-C₂₄ acylisethionates or C₈-C₁₈ acylisethionates. In certain embodiments, the anionic surfactant is chosen from laurylsulfosuccinates.

Carboxylate anionic surfactants comprise at least one carboxylic or carboxylate function (—OOH or —COO⁻) and may optionally also comprise one or more sulfate and/or sulfonate functions. The carboxylic anionic surfactants that may be used thus comprise at least one carboxylic or carboxylate function (—OOH or —COO⁻). They may be chosen from the following compounds: acylglycinates, acyllactylates, acylsarcosinates, acylglutamates, alkyl-D-galactosideuronic acids, alkyl or alkenyl ether carboxylic acids, alkyl(C₆₋₃₀) or alkenyl aryl ether carboxylic acids, alkylamido or alkenylamido ether carboxylic acids; and also the salts of these compounds; the alkyl, alkenyl and/or acyl groups of these compounds comprising up to 30 carbon atoms, such as from 6 to 30 carbon atoms, especially from 8 to 28, better still from 8 to 22 or even from 8 to 18 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

In certain embodiments, C₆-C₂₄ or C₈-C₁₈ alkyl or alkenyl monoesters of polyglycosidepolycarboxylic acids, such as C₆-C₂₄ or C₈-C₁₈ alkyl or alkenyl polyglycoside-citrates, C₆-C₂₄ or C₈-C₁₈ alkyl polyglycoside-tartrates, C₆-C₂₄ or C₈-C₁₈ alkyl or alkenyl polyglycoside-sulfosuccinates, and salts thereof, may be chosen.

In further embodiments, polyoxyalkylenated alkyl(amido) or alkenyl(amido) ether carboxylic acids and salts thereof, in particular those comprising from 2 to 50 alkylene oxide and in particular ethylene oxide groups, such as the compounds sold by the company Kao under the name Akypo, may be chosen. For example, polyoxyalkylenated alkyl(amido) or alkenyl(amido) ether carboxylic acids of formula (XI) may be chosen:

R₁—(OC₂H₄)ₙ—OCH₂COOA (XI)
wherein:
R₁ represents a linear, branched, or cyclic C₅-C₂₄ alkyl or alkenyl radical, optionally substituted, an alkyl(C₈-C₉)phenyl radical, a radical R₂CONH—CH₂—CH₂— with R₂ denoting a linear or branched C₉-C₂₁ alkyl or alkenyl radical, preferably, R₁ is a C₈-C₂₀ and preferably C₈-C₁₈ alkyl radical, and aryl preferably denotes phenyl;
n is an integer or decimal number (average value) ranging from 2 to 24 and preferably from 2 to 10; and
A denotes H, ammonium, Na, K, Li, Mg, or a monoethanolamine or triethanolamine residue.

In certain embodiments, the polyoxyalkylenated alkyl(amido) or alkenyl(amido) ether carboxylic acids of formula (XI) may be those where R₁ is chosen from a C₁₂-C₁₄ alkyl, cocoyl, oleyl, nonylphenyl or octylphenyl radical; A is chosen from a hydrogen or sodium atom, and n ranges from 2 to 20, preferably 2 to 10. In further embodiments, the polyoxyalkylenated alkyl(amido) or alkenyl(amido) ether carboxylic acids of formula (XI) may be those where R₁ is chosen from a C₁₂ alkyl radical; A is chosen from a hydrogen or sodium atom, and n ranges from 2 to 10.

In certain embodiments, the carboxylic anionic surfactant may be chosen from acylglutamates, especially of C₆-C₂₄ or even C₁₂-C₂₀, such as stearoylglutamates, and in particular disodium stearoylglutamate, acylsarcosinates, especially of C₆-C₂₄ or even C₁₂-C₂₀, such as palmitoylsarcosinates, and in particular sodium palmitoylsarcosinate, acyllactylates, especially of C₁₂-C₂₈ or even C₁₄-C₂₄, such as behenoyllactylates, and in particular sodium behenoyllactylate, C₆-C₂₄ and especially C₁₂-C₂₀ acylglycinates, (C₆-C₂₄)alkyl ether carboxylates and especially (C₁₂-C₂₀)alkyl ether carboxylates, and polyoxyalkylenated (C₆-C₂₄)alkyl(amido) ether carboxylic acids, in particular those comprising from 2 to 50 ethylene oxide groups.

As noted herein, the anionic surfactant may optionally be in salt form. In that case, the salt may, for example, be chosen from alkali metal salts, such as the sodium or potassium salt, ammonium salts, amine salts and in particular amino alcohol salts, and alkaline-earth metal salts, such as the magnesium salt. In preferred embodiments, alkali metal or alkaline-earth metal salts may be chosen.

According to the present invention, the anionic surfactant, if it is present, may be present in an amount ranging from about 0.05 wt. % to about 10 wt. %, preferably from about 0.1 wt. % to about 5 wt. %, or from about 0.5 wt. % to about 2 wt. %, relative to the total weight of the colorant composition.

### Nonionic Surfactants

The nonionic surfactant selected from the group consisting of polyoxyalkylenated and polyglycerolated nonionic surfactants are particularly useful. For example, useful nonionic surfactants include hydrogenated castor oil (e.g., PEG-25 Hydrogenated castor oil, PEG-30 Hydrogenated castor oil, PEG-35 Hydrogenated castor oil, PEG-40 Hydrogenated castor oil, PEG-45 Hydrogenated castor oil, PEG-50 Hydrogenated castor oil, PEG-54 Hydrogenated castor oil, PEG-55 Hydrogenated castor oil, PEG-60 Hydrogenated castor oil, PEG-65 Hydrogenated castor oil, PEG-80 Hydrogenated castor oil, PEG-100 Hydrogenated castor oil, and PEG-200 Hydrogenated castor oil), esters of polyols with fatty acids or alkoxylated derivatives thereof (e.g., glyceryl distearate, glyceryl hydroxystearate, glyceryl laurate, glyceryl linoleate, glyceryl myristate, glyceryl oleate, glyceryl stearate, an ethoxylated derivate thereof, or a mixture thereof), and ethoxylated fatty alcohols (or C₈-C₃₀ alcohols).

In some instances, the one or more nonionic surfactants may include PEG-40 hydrogenated castor oil, oleth-5, polysorbate 80, or a mixture thereof.

The nonionic surfactant can be, for example, selected from alcohols, alpha-diols, alkylphenols and esters of fatty acids, these compounds being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropyl-morpholine oxides; and mixtures thereof.

Examples of oxyalkylenated nonionic surfactants that may be mentioned include: oxyalkylenated (C₈-C₂₄)alkylphenols, saturated or unsaturated, linear or branched, oxyalkylenated C₈-C₃₀ alcohols, saturated or unsaturated, linear or branched, oxyalkylenated C₈-C₃₀ amides, esters of saturated or unsaturated, linear or branched, C₈-C₃₀ acids and of polyethylene glycols, polyoxyalkylenated esters of saturated or unsaturated, linear or branched, C₈-C₃₀ acids and of sorbitol, saturated or unsaturated, oxyalkylenated plant oils, condensates of ethylene oxide and/or of propylene oxide, inter alia, alone or as mixtures.

As examples of polyglycerolated nonionic surfactants, polyglycerolated C₈-C₄₀ alcohols may be used. In particular, the polyglycerolated C₈-C₄₀ alcohols correspond to the following formula XII:

RO—[CH₂—CH(CH₂OH)—O]ₘ—H or RO—[CH (CH₂OH)—CH₂O]ₘ—H (XII)
in which R represents a linear or branched C₈-C₄₀ and preferably C₈-C₃₀ alkyl or alkenyl radical, and m represents a number ranging from 1 to 30 and preferably from 1.5 to 10.

As examples of compounds that are suitable in the context of the invention, mention may be made of lauryl alcohol containing 4 mol of glycerol (INCI name: Polyglyceryl-4 Lauryl Ether), lauryl alcohol containing 1.5 mol of glycerol, oleyl alcohol containing 4 mol of glycerol (INCI name: Polyglyceryl-4 Oleyl Ether), oleyl alcohol containing 2 mol of glycerol (INCI name: Polyglyceryl-2 Oleyl Ether), cetearyl alcohol containing 2 mol of glycerol, cetearyl alcohol containing 6 mol of glycerol, oleocetyl alcohol containing 6 mol of glycerol, and octadecanol containing 6 mol of glycerol.

The alcohol may represent a mixture of alcohols in the same way that the value of m represents a statistical value, which means that, in a commercial product, several species of polyglycerolated fatty alcohol may coexist in the form of a mixture.

The nonionic surfactants may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as the compounds with the INCI names: PEG-9 to PEG-50 laurate; PEG-9 to PEG-50 palmitate; PEG-9 to PEG-50 stearate; PEG-9 to PEG-50 palmitostearate; PEG-9 to PEG-50 behenate; and the compound polyethylene glycol 100 EO monostearate (INCI name: PEG-100 stearate); and mixtures thereof.

As glyceryl esters of fatty acids, glyceryl stearate (glyceryl mono-, di- and/or tristearate) (CTFA name: glyceryl stearate) or glyceryl ricinoleate and mixtures thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

The sorbitol esters of C₈-C₂₄ fatty acids and alkoxylated derivatives thereof can be selected from sorbitan palmitate, sorbitan trioleate and esters of fatty acids and alkoxylated sorbitan containing for example from 20 to 100 EO, such as for example polyethylene sorbitan trioleate (polysorbate 85) or the compounds marketed under the trade names Tween 20 or Tween 60 by Ubiqema.

As esters of fatty acids and glucose or alkylglucose, in particular glucose palmitate, alkylglucose sesquistearates such as methylglucose sesquistearate, alkylglucose palmitates such as methylglucose or ethylglucose palmitate, methylglucoside fatty esters and more specifically the diester of methylglucoside and oleic acid (INCI name: Methyl glucose dioleate), the mixed ester of methylglucoside and the mixture oleic acid/hydroxystearic acid (INCI name: Methyl glucose dioleate/hydroxystearate), the ester of methylglucoside and isostearic acid (INCI name: Methyl glucose isostearate), the ester of methylglucoside and lauric acid (INCI name: Methyl glucose laurate), the mixture of monoester and diester of methylglucoside and isostearic acid (INCI name: Methyl glucose sesqui-isostearate), the mixture of monoester and diester of methylglucoside and stearic acid (INCI name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (INCI name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (INCI name: PEG-20 methyl glucose sesquistearate) and in particular the product marketed under the name Glucamate SSE-20 by AMERCHOL and that marketed under the name Grillocose PSE-20 by GOLDSCHMIDT, and mixtures thereof, can for example be cited.

As sucrose esters, saccharose palmito-stearate, saccharose stearate and saccharose monolaurate can for example be cited.

As sugar ethers, alkylpolyglucosides can be used, and for example decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, and mixtures thereof can in particular be cited.

According to the present invention, the nonionic surfactant, if it is present, may be present in an amount ranging from about 0.01 wt. % to about 15 wt. %, preferably from about 0.05 wt. % to about 10 wt. %, or from about 0.1 wt. % to about 5 wt. %, relative to the total weight of the colorant composition.

The surfactant system, comprising at least one surfactant selected from the group consisting of nonionic surfactants which are different from the above liquid polar fatty substance, amphoteric surfactants, anionic surfactants and mixtures thereof may be present in an amount ranging from about 0.1 wt. % to about 30 wt. %, preferably from about 0.5 wt. % to about 20 wt. %, or from about 1 wt. % to about 15 wt. %, more preferably from about 2 wt. % to about 10 wt. % relative to the total weight of the colorant composition.

### Solvent

Generally, the colorant composition of the present invention may comprise at least one solvent. The solvent of the colorant composition of the present invention may include one or more water-miscible or at least partially water-miscible compounds (at room temperature of 20-25° C.), for instance C₂-C₈ lower polyols, monoalcohols, or polyol ethers (especially containing from 3 to 16 carbon atoms). The solvent of the colorant composition of the present invention may include water, or even consist essentially of water.

Preferably, the composition according to the invention comprises water.

Advantageously, the solvent is present in an amount ranging from about 15 wt. % to about 80 wt. %, preferably from about 20 wt. % to about 70 wt. %, or from about 25 wt. % to about 60 wt. %, relative to the total weight of the colorant composition.

Advantageously, the water is present in an amount ranging from 15 wt. % to 80 wt. %, preferably from 20 wt. % to 70 wt. %, or from 25 wt. % to 60 wt. %, relative to the total weight of the colorant composition.

### Other Ingredients

The colorant composition according to the present invention may also comprise other ingredients, known previously elsewhere in cosmetic compositions, such as alkalizing agents, antioxidants, fragrances, and so on.

One non-limiting example of the alkalizing agent that can be mentioned is monoethanolamine.

Examples of the antioxidant that can be mentioned include, but not limit to ascorbic acid, sodium metabisulfite, and a mixture thereof.

Advantageously, the alkalizing agent is present in an amount ranging from about 1 wt. % to about 25 wt. %, preferably from about 5 wt. % to about 15 wt. %, relative to the total weight of the colorant composition.

Advantageously, the antioxidant is present in an amount ranging from about 0.1 wt. % to about 5 wt. %, preferably from about 0.5 wt. % to about 3 wt. %, relative to the total weight of the colorant composition.

### Developer Composition

The developer composition according to the present invention may comprise at least one nonionic surfactant, at least one oxidizing agent, and at least one solvent.

Preferably, the nonionic surfactant may be chosen from fatty alcohol-based compounds, fatty amide-based compounds, and mixtures thereof.

The term “fatty alcohol-based compounds” according to the present invention includes fatty alcohols, oxyalkylenated fatty alcohols, and mixtures thereof. The term “fatty alcohol” means a long-chain aliphatic alcohol comprising from 8 to 30 carbon atoms and comprising at least one hydroxyl group OH.

Examples of the fatty alcohol-based compounds that can be mentioned include, but not limit to cetearyl alcohol, which is a mixture of cetyl alcohol and stearyl alcohol, stearyl alcohol 20 OE (CTFA name steareth-20), cetearyl alcohol 25 OE (CTFA name ceteareth-25) and mixtures thereof.

The fatty amide-based compounds according to the present invention are chosen from oxyalkylenated fatty amides, which are chosen from the compounds of formula (XIII) below:

R—CO—N(R′)-(Alk-O)ₙH (XIII)
wherein:
R denotes an optionally substituted C₈-C₃₀, preferably C₁₀-C₂₄ and better still C₁₂-C₂₂ alkyl or alkenyl radical,
R′ denotes a hydrogen atom or an (Alk-O)ₘH radical, and preferably a hydrogen atom,
Alk denotes a divalent alkylene radical comprising from 1 to 8 carbon atoms, preferably 2 or 3 carbon atoms,
n, m denote, independently of one another, a number ranging from 1 to 50, preferably from 1 to 20, better still from 1 to 10.

One non-limiting example of fatty amide-based compounds that can be mentioned is the compound having the INCI name PEG-4 rapeseedamide, sold in particular under the name Amidet®N by the company Kao.

According to the present invention, the nonionic surfactant is present in an amount ranging from about 0.1 wt. % to about 20 wt. %, preferably from about 0.5 wt. % to about 15 wt. %, or from about 1 wt. % to about 10 wt. %, relative to the total weight of the developer composition.

### Oxidizing Agents

The oxidizing agent according to the present invention is chosen from hydrogen peroxide and/or one or more hydrogen peroxide-generating systems.

Specifically, the oxidizing agent is selected from the group consisting of hydrogen peroxide, persalts such as persulphates, percarbonates and perborates, urea peroxide, and mixtures thereof. Preferably, the oxidizing agent is chosen from hydrogen peroxide.

According to the present invention, the oxidizing agent is present in an amount ranging from about 0.1 wt. % to about 25 wt. %, preferably from about 1 wt. % to about 20 wt. %, or from about 5 wt. % to about 15 wt. %, relative to the total weight of the developer composition.

### Solvent

The developer composition according to the invention may comprise at least one solvent. The useful solvent can be selected from those as defined for the “solvent” of the above colorant composition.

Advantageously, the solvent is present in an amount ranging from about 30 wt. % to about 95 wt. %, preferably from about 40 wt. % to about 90 wt. %, relative to the total weight of the developer composition.

### Other Ingredients

The developer composition according to the present invention may also comprise other ingredients, known previously elsewhere in cosmetic compositions, such as chelating agents, preservatives, fragrances, and so on.

Examples of the chelating agent that can be mentioned include, but not limit to tetrasodium etidronate, tetrasodium pyrophosphate, and mixtures thereof.

One non-limiting example of the preservative that can be mentioned is Sodium Salicylate.

Advantageously, the chelating agent is present in an amount ranging from about 0.01 wt. % to about 2 wt. %, preferably from about 0.1 wt. % to about 1 wt. %, relative to the total weight of the developer composition.

Advantageously, the preservative is present in an amount ranging from about 0.001 wt. % to about 1 wt. %, preferably from about 0.01 wt. % to about 0.1 wt. %, relative to the total weight of the developer composition.

### Dye Kit

In another aspect, the present invention relates to a dye kit, comprising a colorant composition and a developer composition, wherein both the colorant composition and the developer composition are defined above.

The colorant composition and/or the developer composition of the present invention are in the form of cream. In one embodiment, both the colorant composition and the developer composition of the present invention are in the form of cream.

Conventionally, cream products show good color performance but may not be convenient enough to apply, while foam products in the market are convenient to use but the color performance may not be good enough.

By means of the specific colorant composition comprising the above oxidative dye, hydrophilic gelling polymer, surfactant system, and liquid polar fatty substance of the present invention, even in the form of cream, the mixture from the colorant composition and the developer composition of the present invention can still provide easy application similar with foam products and better color performance than foam products, i.e. as good color performance as traditional cream products.

In one embodiment, both the colorant composition and the developer composition of the present invention are ammonia-free compositions, i.e. they do not comprise the ingredients which can generate ammonia, e.g. ammonium hydroxide, ammonium bicarbonate, and ammonium persulfate, such that the mixture from the colorant composition and the developer composition of the present invention would not generate unpleasant smell when being applied.

In one embodiment, the colorant composition and the developer composition of the present invention are disposed in two different compartments respectively. For example, the colorant composition and the developer composition are disposed in two different pouches or bottles for the dye kit of the present invention.

In one embodiment, the dye kit of the present invention is equipped with means allowing the delivery to the hair of the mixture of the colorant composition and the developer composition, for example, the device described in patent FR 2 586 913.

### Process

In another aspect, the present invention relates to a process for dyeing keratin fibers, in particular the hair, using the dye kit as described above.

According to a preferred embodiment, the dyeing process of the invention comprises mixing the colorant composition and the developer composition immediately before use, and applying the mixture obtained as described above to the keratin fibers.

In one embodiment, the colorant composition of the present invention is put into a container or palm together with the developer composition as described above, with or without stirring them.

The mixture of the colorant composition and the developer composition is usually left in place on the keratin fibers for a time generally ranging from 1 minute to 1 hour and preferably from 5 minutes to 30 minutes.

The temperature during the dyeing process is conventionally between 2° and 80° C. and preferably between 2° and 60° C. After the treatment, the human keratin fibers are advantageously rinsed with water. They may optionally be further washed with a shampoo, followed by rinsing with water, before being dried or left to dry.

The process may be repeated several times in order to obtain the desired coloration.

The present invention is illustrated in greater detail by the examples described below, which are given as non-limiting illustrations.

### EXAMPLES

Main raw materials used, trade names and supplier thereof were listed in Table 1.

TABLE 1

| INCI Name | Trade Name | Supplier |
|---|---|---|
| PEG-40 HYDROGENATED | Kolliphor ® RH 40 | BASF |
| CASTOR OIL | | |
| COCAMIDOPROPYL BETAINE | DEHYTON PK 45 | BASF |
| SODIUM LAURYL SULFATE | Kolliphor ® SLS Fine | BASF |
| WATER | EAUDESIONISEE | USINE |
| | MICROBIOLOGIQUEMENT | L'OREAL |
| | PROPRE | |
| ACRYLATES COPOLYMER | Carbopol ® Aqua SF-1 | LUBRIZOL |
| ETHYLHEXYL PALMITATE | CEGESOFT C 24 | BASF |
| ASCORBIC ACID | ASCORBIC ACID 100 | CSPC |
| | MESH | WEISHENG |
| | | PHARMACEUTICAL |
| SODIUM METABISULFITE | SODIUM | ESSECO |
| | METABISULPHITE HP | |
| ETHANOLAMINE | MONOETHANOLAMINE | BASF |
| | CARE | |
| HYDROXYBENZOMORPHOLINE | 3,4-DIHYDRO-2H-1,4-BENZOXAZIN- | DRAGON |
| | 6-OL(IMEXINEOV) | CHEMICAL |
| 2,4-DIAMINOPHENOXYETHANOL | 24DAPE LO-BLEU | DRAGON |
| HCl | | CHEMICAL |
| N,N-BIS(2-HYDROXYETHYL)-p-PH | LO NNBIS | DRAGON |
| ENYLENEDIAMINE SULFATE | | CHEMICAL |
| m-AMINOPHENOL | RODOL EG | LOWENSTEIN |
| 2-METHOXYMETHYL-P- | 2-METHOXY-METHYL-P- | DRAGON |
| PHENYLENEDIAMINE | PHENYLENEDIAMINE | CHEMICAL |
| HYDROXYPROPYL GUAR | JAGUAR HP 105 SGI | RHODIA |
| | | (SOLVAY) |
| XANTHAN GUM | KELTROL CG-T | CP KELCO |
| HYDROXYETHYLCELLULOSE | NATROSOL 250 HHR CS | ASHLAND |
| PEG-4 RAPESEEDAMIDE | AMIDET N | KAO |
| CETEARYL ALCOHOL | ECOROL 68/30 P/MB | ECOGREEN |
| | | OLEOCHEMICALS |
| CETEARETH-25 | EUMULGIN B 25 | BASF |
| STEARETH-20 | ALKAMULS AL S20 MB | RHODIA |
| | | (SOLVAY) |
| TETRASODIUM PYROPHOSPHATE | SODIUM | JIANGSU |
| | PYROPHOSPHATE | KOLOD FOOD |
| | (DECAHYDRATE) | INGREDIENTS |
| SODIUM SALICYLATE | SALICYLATE DE | API |
| | SODIUM | CORPORATION |
| | (PHARMACEUTICAL | |
| | GRADE) | |
| TETRASODIUM ETIDRONATE | TURPINAL 4 NL | ITALMATCH |
| | | CHEMICALS |
| HYDROGEN PEROXIDE | H₂O₂ INTEROX ST-50 | SOLVAY |
| ISOPROPYL MYRISTATE | ISOPROPYLMYRISTATE | BASF |
| ISOPROPYL PALMITATE | ISOPROPYL PALMITATE | BASF |
| MINERAL OIL | AEMOIL M-302CG | AEMEDSA |
| ISODODECANE | ISODODECANE | INEOS |
| PHOSPHORIC ACID | PRAYPHOS P5 85% | PRAYON |

### Inventive Examples 1-5 and Comparative Examples 1-9

The colorant compositions according to inventive formula Exs. 1-5 and comparative formulas CEs. 1-9 were prepared with the ingredients listed in Tables 2-4 (the contents were expressed as weight percentages of ingredients with regard to the total weight of each composition, unless otherwise indicated):

TABLE 2

| Components | Ex. 1 | Ex. 2 | Ex. 3 | Ex. 4 | Ex. 5 |
|---|---|---|---|---|---|
| ACRYLATES COPOLYMER | 2.4 | 2.4 | 2.4 | 2.4 | 2.4 |
| ETHANOLAMINE | 9.9 | 9.9 | 9.9 | 9.9 | 9.9 |
| HYDROXYBENZOMORPHOLINE | 2.0 | 2.0 | 2.0 | 2.0 | 2.0 |
| ETHYLHEXYL PALMITATE | 30.0 | 20.0 | 40.0 | / | / |
| ISOPROPYL MYRISTATE | / | / | / | 30.0 | / |
| ISOPROPYL PALMITATE | / | / | / | / | 30.0 |
| 2-METHOXYMETHYL-P- | 2.8 | 2.8 | 2.8 | 2.8 | 2.8 |
| PHENYLENEDIAMINE | | | | | |
| ASCORBIC ACID | 0.3 | 0.3 | 0.3 | 0.3 | 0.3 |
| N,N-BIS(2-HYDROXYETHYL)-P- | 0.82 | 0.82 | 0.82 | 0.82 | 0.82 |
| PHENYLENEDIAMINE | | | | | |
| SULFATE | | | | | |
| m-AMINOPHENOL | 0.6 | 0.6 | 0.6 | 0.6 | 0.6 |
| PEG-40 HYDROGENATED | 1.0 | 1.0 | 1.0 | 1.0 | 1.0 |
| CASTOR OIL | | | | | |
| COCAMIDOPROPYL BETAINE | 3.8 | 3.8 | 3.8 | 3.8 | 3.8 |
| 2,4-DIAMINOPHENOXYETHANOL | 0.6 | 0.6 | 0.6 | 0.6 | 0.6 |
| HCL | | | | | |
| SODIUM METABISULFITE | 0.7 | 0.7 | 0.7 | 0.7 | 0.7 |
| SODIUM LAURYL SULFATE | 0.7 | 0.7 | 0.7 | 0.7 | 0.7 |
| WATER | QS100 | QS100 | QS100 | QS100 | QS100 |

TABLE 3

| Components | CE. 1 | CE. 2 | CE. 3 | CE. 4 |
|---|---|---|---|---|
| HYDROXYPROPYL GUAR | 2.4 | / | / | 1.2 |
| XANTHAN GUM | / | 2.4 | / | / |
| HYDROXYETHYLCELLULOSE | / | / | 2.4 | 1.2 |
| ETHANOLAMINE | 9.9 | 9.9 | 9.9 | 9.9 |
| HYDROXYBENZOMORPHOLINE | 2.0 | 2.0 | 2.0 | 2.0 |
| ETHYLHEXYL PALMITATE | 30.0 | 30.0 | 30.0 | 30.0 |
| 2-METHOXYMETHYL-P- | 2.8 | 2.8 | 2.8 | 2.8 |
| PHENYLENEDIAMINE | | | | |
| ASCORBIC ACID | 0.3 | 0.3 | 0.3 | 0.3 |
| N,N-BIS(2-HYDROXYETHYL)-P- | 0.82 | 0.82 | 0.82 | 0.82 |
| PHENYLENEDIAMINE SULFATE | | | | |
| m-AMINOPHENOL | 0.6 | 0.6 | 0.6 | 0.6 |
| PEG-40 HYDROGENATED | 1.0 | 1.0 | 1.0 | 1.0 |
| CASTOR OIL | | | | |
| COCAMIDOPROPYL BETAINE | 3.8 | 3.8 | 3.8 | 3.8 |
| 2,4-DIAMINOPHENOXYETHANOL | 0.6 | 0.6 | 0.6 | 0.6 |
| HCL | | | | |
| SODIUM METABISULFITE | 0.7 | 0.7 | 0.7 | 0.7 |
| SODIUM LAURYL SULFATE | 0.7 | 0.7 | 0.7 | 0.7 |
| WATER | QS100 | QS100 | QS100 | QS100 |

TABLE 4

| Components | CE. 5 | CE. 6 | CE.7 | CE.8 | CE.9 |
|---|---|---|---|---|---|
| ACRYLATES COPOLYMER | 2.4 | 2.4 | 2.4 | 2.4 | 2.4 |
| ETHANOLAMINE | 9.9 | 9.9 | 9.9 | 9.9 | 9.9 |
| HYDROXYBENZOMORPHOLINE | 2.0 | 2.0 | 2.0 | 2.0 | 2.0 |
| ETHYLHEXYL PALMITATE | / | / | 5.0 | 60.0 | / |
| MINERAL OIL | 30.0 | / | / | / | / |
| ISODODECANE | / | 30.0 | / | / | / |
| CETEARYL ALCOHOL | / | / | / | / | 30.0 |
| 2-METHOXYMETHYL-P- | 2.8 | 2.8 | 2.8 | 2.8 | 2.8 |
| PHENYLENEDIAMINE | | | | | |
| ASCORBIC ACID | 0.3 | 0.3 | 0.3 | 0.3 | 0.3 |
| N,N-BIS(2-HYDROXYETHYL)-P- | 0.82 | 0.82 | 0.82 | 0.82 | 0.82 |
| PHENYLENEDIAMINE | | | | | |
| SULFATE | | | | | |
| m-AMINOPHENOL | 0.6 | 0.6 | 0.6 | 0.6 | 0.6 |
| PEG-40 HYDROGENATED | 1.0 | 1.0 | 1.0 | 1.0 | 1.0 |
| CASTOR OIL | | | | | |
| COCAMIDOPROPYL BETAINE | 3.8 | 3.8 | 3.8 | 3.8 | 3.8 |
| 2,4-DIAMINOPHENOXYETHANOL | 0.6 | 0.6 | 0.6 | 0.6 | 0.6 |
| HCL | | | | | |
| SODIUM METABISULFITE | 0.7 | 0.7 | 0.7 | 0.7 | 0.7 |
| SODIUM LAURYL SULFATE | 0.7 | 0.7 | 0.7 | 0.7 | 0.7 |
| WATER | QS100 | QS100 | QS100 | QS100 | QS100 |

Notes:
Mineral oil has a polarity index value falling in the range of 38.3-43.7 mN/m, Isododecane has a polarity index value of 53 mN/m, wherein the polarity index means the polarity or surface tension (in 10⁻³ Newton/meter), as measured by the ring method using a ring tensiometer at 20° C against air, and Cetearyl alcohol is solid (rather than liquid) at room temperature and atmospheric pressure.

A developer composition hereinafter was prepared with the ingredients listed in Table 5 (the contents were expressed as weight percentages of ingredients with regard to the total weight of the developer composition, unless otherwise indicated).

TABLE 5

| Components | Content |
|---|---|
| CETEARYL ALCOHOL | 2.3 |
| STEARETH-20 | 0.2 |
| PEG-4 RAPESEEDAMIDE | 1.1 |
| CETEARETH-25 | 0.6 |
| SODIUM SALICYLATE | 0.04 |
| HYDROGEN PEROXIDE | 9.0 |
| TETRASODIUM ETIDRONATE | 0.2 |
| TETRASODIUM PYROPHOSPHATE | 0.04 |
| WATER | QS100 |

### Preparation Procedure:

The colorant compositions were prepared as follows:
1). adding water and Acrylates Copolymer/Hydroxypropyl Guar/Xanthan Gum/Hydroxyethylcellulose into the first main kettle and stirring the mixture at room temperature at the speed of 150-200 rpm for 5 minutes until the mixture was uniform;
2). adding Sodium Lauryl Sulfate, Cocamidopropyl Betaine, and PEG-40 Hydrogenated Castor Oil into the first main kettle and stirring the mixture at room temperature at the speed of 200-300 rpm for 15 minutes until the mixture was uniform;
3). adding Ethanolamine into the first main kettle and stirring the mixture at room temperature at the speed of 200-300 rpm for 5-10 minutes until the mixture was uniform;
4). adding Ethylhexyl Palmitate/Isopropyl Myristate/Isopropyl Palmitate/Mineral oil/Isododecane/Cetearyl alcohol into the first main kettle and stirring the mixture at room temperature at the speed of 600-1000 rpm for 15-20 minutes to emulsify the mixture; and
5). adding Ascorbic Acid, Sodium Metabisulfite, Hydroxybenzomorpholine, m-Aminophenol, 2-Methoxymethyl-p-phenylenediamine, N,N-bis(2-hydroxyethyl)-p-phenylenediamine Sulfate, and 2,4-Diaminophenoxyethanol HCl into the first main kettle and stirring the mixture at room temperature at the speed of 600-1000 rpm for 15 minutes until the mixture was uniform.

The developer compositions were prepared as follows:
1). adding 80% of the water, Cetearyl Alcohol, Steareth-20, PEG-4 Rapeseedamide, Ceteareth-25, Sodium Salicylate, Tetrasodium Etidronate, and Tetrasodium Pyrophosphate into the second main kettle and stirring and heating the mixture to the temperature of 75° C. at the speed of 200-300 rpm; after the temperature reached 75° C., stirring and emulsifying the mixture at the speed of 300-600 rpm for 15 minutes, and then cooling the mixture to 45° C.;
2). adding the rest of water (20%) into the second main kettle, stirring the mixture at the speed of 400-600 rpm for 10 minutes, and then cooling the mixture to 35° C.;
3). adding Hydrogen Peroxide into the second main kettle, cooling and stirring the mixture at the speed of 400-600 rpm for 5-10 minutes until the mixture was uniform, and then adjusting the mixture to the pH value of 2.2+−0.2 using phosphoric acid.

### Formula Stability Test

The colorant compositions of Inventive Example 1-5 and Comparative Examples 1-9 were tested for stability as follows:
1). preparing the colorant compositions respectively according to the above processes, and then aging for 24 hours at room temperature;
2). filling the compositions into respective packages and putting them under the following conditions in an oven or refrigerator: 2 weeks, 50° C.; 1 month, room temperature/4° C./45° C.; 2 months, room temperature/45° C.;
3). checking appearance and stability results of the compositions after 2 weeks/1 month/2 months.

The results of appearance and stability were summarized in Tables 6-8.

TABLE 6

| Properties | Ex. 1 | Ex. 2 | Ex. 3 | Ex. 4 | Ex. 5 |
|---|---|---|---|---|---|
| Stability and | Good, | Good, | Good, | Good, | Good, |
| Appearance | Uniform | Uniform | Uniform | Uniform | Uniform |
| (after being | | | | | |
| stored at 4° C. | | | | | |
| for 1 month) | | | | | |
| Stability and | Good, | Good, | Good, | Good, | Good, |
| Appearance | Uniform | Uniform | Uniform | Uniform | Uniform |
| (after being | | | | | |
| stored at | | | | | |
| room temperature | | | | | |
| for 1 month) | | | | | |
| Stability and | Good, | Good, | Good, | Good, | Good, |
| Appearance | Uniform | Uniform | Uniform | Uniform | Uniform |
| (after being | | | | | |
| stored at | | | | | |
| room temperature | | | | | |
| for 2 months) | | | | | |
| Stability and | Good, | Good, | Good, | Good, | Good, |
| Appearance | Uniform | Uniform | Uniform | Uniform | Uniform |
| (after being | | | | | |
| stored at | | | | | |
| 50° C. for | | | | | |
| 2 weeks) | | | | | |
| Stability and | Good, | Good, | Good, | Good, | Good, |
| Appearance | Uniform | Uniform | Uniform | Uniform | Uniform |
| (after being | | | | | |
| stored at | | | | | |
| 45° C. for | | | | | |
| 1 month) | | | | | |
| Stability and | Good, | Good, | Good, | Good, | Good, |
| Appearance | Uniform | Uniform | Uniform | Uniform | Uniform |
| (after being | | | | | |
| stored at | | | | | |
| 45° C. for | | | | | |
| 2 months) | | | | | |

TABLE 7

| Properties | CE. 1 | CE. 2 | CE. 3 | CE. 4 |
|---|---|---|---|---|
| Stability and Appearance | Good, | Good, | Good, | Good, |
| (after being stored | Uniform | Uniform | Uniform | Uniform |
| at 4° C. for 1 month) | | | | |
| Stability and Appearance | Good, | Good, | Good, | Good, |
| (after being stored | Uniform | Uniform | Uniform | Uniform |
| at room temperature | | | | |
| for 1 month) | | | | |
| Stability and Appearance | Good, | Good, | Good, | Good, |
| (after being stored | Uniform | Uniform | Uniform | Uniform |
| at room temperature | | | | |
| for 2 months) | | | | |
| Stability and Appearance | Good, | Good, | Good, | Good, |
| (after being stored | Uniform | Uniform | Uniform | Uniform |
| at 50° C. for 2 weeks) | | | | |
| Stability and Appearance | Good, | Good, | Good, | Good, |
| (after being stored | Uniform | Uniform | Uniform | Uniform |
| at 45° C. for 1 month) | | | | |
| Stability and Appearance | Poor, | Poor, | Poor, | Poor, |
| (after being stored | Phase | Phase | Phase | Phase |
| at 45° C. for 2 months) | Separation | Separation | Separation | Separation |

TABLE 8

| Properties | CE. 5 | CE. 6 | CE.7 | CE.8 | CE.9 |
|---|---|---|---|---|---|
| Stability and Appearance | Good, | Good, | Poor, | Unable to | Good, |
| (after being stored | Uniform | Uniform | Phase | prepare | Uniform |
| at 4° C. for 1 month) | | | Separation | formula | |
| Stability and Appearance | Good, | Good, | Good, | Unable to | Good, |
| (after being stored | Uniform | Uniform | Uniform | prepare | Uniform |
| at room temperature | | | | formula | |
| for 1 month) | | | | | |
| Stability and Appearance | Good, | Poor, | Poor, | Unable to | Good, |
| (after being stored | Uniform | Phase | Phase | prepare | Uniform |
| at room temperature | | Separation | Separation | formula | |
| for 2 months) | | | | | |
| Stability and Appearance | Good, | Poor, | Poor, | Unable to | Good, |
| (after being stored | Uniform | Phase | Phase | prepare | Uniform |
| at 50° C. for 2 weeks) | | Separation | Separation | formula | |
| Stability and Appearance | Good, | Poor, | Poor, | Unable to | Good, |
| (after being stored | Uniform | Phase | Phase | prepare | Uniform |
| at 45° C. for 1 month) | | Separation | Separation | formula | |
| Stability and Appearance | Good, | Poor, | Poor, | Unable to | Poor, |
| (after being stored | | Phase | Phase | prepare | Phase |
| at 45° C. for 2 months) | Uniform | Separation | Separation | formula | Separation |

Notes:
“Unable to prepare formula” meant that the composition was unable to get emulsified and phase separation occurred during processing.

From the above Tables 6-8, it could be seen that, the type of polymer and the type of fatty substance and the amount of fatty substance were essential to obtain desirable stability for the colorant compositions comprising the same.

Specifically, only the colorant compositions of Inventive Examples 1-5 could obtain desirable stability after being stored at a lower temperature, e.g. 4° C., at room temperature, and at a higher temperature, e.g. 45° C. or 50° C., for the time as long as 2 months.

By contrast, the colorant compositions of Comparative Examples 1-4, i.e. those comprising polymers out of the inventive ones, obtained poor stability, after being stored at a higher temperature, e.g. 45° C., for a longer time, e.g. 2 months.

Moreover, the colorant composition of Comparative Example 6, i.e. the one comprising a fatty substance having a polarity index value of higher than 26, obtained poor stability, after being stored at a higher temperature, e.g. 45° C. or 50° C., or at room temperature for a longer time, e.g. 2 months. The colorant composition of the comparative example 7 comprising an amount of less than 10 wt. % of the inventive fatty substances obtained poor stability after being stored at a lower or higher temperature, e.g. 4° C., 45° C. or 50° C., or at room temperature for a longer time, e.g. 2 months, wherein the colorant composition of Comparative Example 8, i.e. the one comprising 60 wt. % of the inventive fatty substance, was even unable to form a formula. The colorant composition of the Comparative Example 9, i.e. the one comprising a solid fatty substance, also obtained poor stability, after being stored at a higher temperature, e.g. 45° C., for a longer time, e.g. 2 months.

### Easy Application Test

Each of the colorant compositions of Inventive Example 1-5 and Comparative Examples 1-9 and the developer composition as shown in Table 5 were mixed at a ratio of 1:1 by weight. The time consumed to evenly apply the mixtures on half mannequin head by hands were recorded and compared, wherein each mixture was applied twice by different internal hair experts, and then the average time were calculated and the scores were given.

The scores were judged by application time and summarized in Tables 9-10.

Specifically, <100 sec→10
100~110 sec→9
110~120 sec→8
120~130 sec→7
130~140 sec→6
140~150 sec→5
150~160 sec→4
160~170 sec→3
170~180 sec→2
>=180 sec→1.

TABLE 9

| Properties | Ex. 1 | Ex. 2 | Ex. 3 | Ex. 4 | Ex. 5 |
|---|---|---|---|---|---|
| Application | 9 | 9 | 9 | 9 | 9 |
| scores | | | | | |
| (1~10) | | | | | |

TABLE 10

| Properties | CE. 1 | CE. 2 | CE. 3 | CE. 4 | CE. 5 | CE. 6 | CE. 7 | CE. 8 | CE. 9 |
|---|---|---|---|---|---|---|---|---|---|
| Application scores | 2 | 3 | 2 | 2 | 5 | 5 | 9 | / | 1 |
| (1~10) | | | | | | | | | |

From the above Tables 9-10, it could be seen that, each of the mixtures from the colorant compositions of Inventive Examples 1-5 and the developer composition as shown in Table 5 provided the application score of 9, while most of the mixtures from the colorant compositions of Comparative Examples 1-9 and the same developer composition provided the application scores of 5 or less. In other words, the mixtures from the colorant compositions of Inventive Examples 1-5 and the developer composition were applied much easier than the mixtures from the colorant compositions of most of the comparative examples and the same developer composition.

### Dyeing Performance Evaluation

The dyeing performance obtained from Inventive Example 1-5 and Comparative Examples 1-9 were evaluated as follows:
1). preparing the dyeing mixture by mixing 5 g of each of the colorant compositions with 5 g of the developer composition.
2). on a hot plate of 27° C., preparing 1 g fiber swatch of Chinese 100% grey hair and then dyeing the hair swatch using brush with the dyeing mixtures as prepared above, wherein every hair fiber was covered by the mixture.
3). rinsing off the hair swatch with water and shampoo after 30 minutes, then drying the hair swatch with hair dryer.
4). evaluating the dyeing performance by comparing hair color before and after dyeing in certain light box by hair color experts, then giving scores based on internal technical hair color tone level and reflect.

The results of dyeing performance evaluation were summarized in Tables 11-12.

TABLE 11

| Properties | Ex. 1 | Ex. 2 | Ex. 3 | Ex. 4 | Ex. 5 |
|---|---|---|---|---|---|
| Dyeing performance | Very | Good | Very | Very | Very |
| | good | | good | good | good |
| Internal score (1~10) | 9 | 8 | 9 | 9 | 9 |

TABLE 12

| Properties | CE. 1 | CE. 2 | CE. 3 | CE. 4 | CE. 5 | CE. 6 | CE. 7 | CE. 8 | CE. 9 |
|---|---|---|---|---|---|---|---|---|---|
| Dyeing | Very | Very | Very | Very | Very | Very | Poor | / | Good |
| performance | good | good | good | good | good | good | | | |
| Internal score | 9 | 9 | 9 | 9 | 9 | 9 | 5 | / | 8 |
| (1~10) | | | | | | | | | |

From the above Tables 11-12, it could be seen that, the mixtures from the colorant compositions of Inventive Examples 1-5 and the developer composition as shown in Table 5 provided good or very good dyeing performance. By contrast, the mixtures from the colorant compositions of Comparative Examples comprising an amount of less than 10 wt. % or higher than 50 wt. % of the inventive fatty substances and the same developer composition obtained poor dyeing performance, wherein the colorant composition of Comparative Example 8 was even unable to form a formula as mentioned above.

In sum, it could be seen that only the colorant compositions of Inventive Examples 1-5 had good stability and provided easy application and good dyeing performance.

1: A colorant composition for dyeing keratin fibers, comprising
(i) at least one oxidative dye chosen from oxidation bases, optionally in combination with one or more couplers;
(ii) at least one hydrophilic gelling polymer chosen from anionic acrylic copolymers;
(iii) from 10 wt. % to 50 wt. % of at least one liquid polar fatty substance having a polarity index value of less than 26 mN/m, relative to the total weight of the colorant composition; and
(iv) a surfactant system, comprising at least one surfactant selected from the group consisting of nonionic surfactants which are different from said liquid polar fatty substance, amphoteric surfactants, anionic surfactants and mixture thereof.

2: The colorant composition according to claim 1, wherein the composition is in the form of cream; and/or the surfactant system comprises at least one amphoteric surfactant, at least one anionic surfactant and at least one nonionic surfactant.

3: The colorant composition according to claim 1, wherein the oxidation bases are chosen from p-phenylenediamines, bis(phenyl)alkylenediamines, p-aminophenols, o-aminophenols, heterocyclic bases, and the addition salts thereof, and mixtures thereof, and/or
the couplers are chosen from m-phenylenediamines, m-aminophenols, m-diphenols, naphthalene-based couplers, heterocyclic couplers, and also the addition salts thereof, and mixtures thereof.

4: The colorant composition according to claim 1, wherein the anionic acrylic copolymers are chosen from
anionic copolymers derived from at least one unsaturated carboxylic acid and from at least one ester of an unsaturated carboxylic acid and of a monoalcohol comprising from 1 to 6 carbon atoms;
anionic associative acrylic copolymers, chosen from copolymers of an α,β-monoethylenically unsaturated carboxylic acid, of an ester of an α,β-monoethylenically unsaturated carboxylic acid and of a polyoxyethylenated C₁₂-C₃₀ fatty alcohol; and
mixtures thereof.

5: The colorant composition according to claim 1, wherein the amphoteric surfactants are chosen from betaines, alkyl sultaines, alkyl amphoacetates, alkyl amphoproprionates, and mixtures thereof;
the anionic surfactants are chosen from sulfate, sulfonate, carboxylic or carboxylate surfactants, and mixtures thereof; and/or
the nonionic surfactants are chosen from polyoxyalkylenated and polyglycerolated hydrogenated castor oils, glyceryl esters of fatty acids, glyceryl esters of C₈-C₂₄ alkoxylated fatty acids, esters of fatty acids and glucose or alkylglucose, ethoxylated ethers of fatty acids and glucose or alkylglucose, alkylpolyglucosides, and mixtures thereof.

6: The colorant composition according to claim 1, wherein the liquid polar fatty substance is chosen from fatty alcohols, esters of fatty acid, esters of fatty alcohol, oils, silicones and mixtures thereof.

7: The colorant composition according to claim 1, wherein the oxidation bases are present in an amount ranging from 0.1 wt. % to 15.0 wt. %, relative to the total weight of the colorant composition, and/or
the couplers are present in an amount ranging from 0.1 wt. % to 15.0 wt. %, relative to the total weight of the colorant composition.

8: The colorant composition according to claim 1, wherein the hydrophilic gelling polymer is present in an amount ranging from 0.5 wt. % to 10.0 wt. %, relative to the total weight of the colorant composition.

9: The colorant composition according to claim 1, wherein the amphoteric surfactant is present in an amount ranging from 0.05 wt. % to 20 wt. %, relative to the total weight of the colorant composition;
the anionic surfactant is present in an amount ranging from 0.05 wt. % to 10 wt. %, relative to the total weight of the colorant composition; and/or
the nonionic surfactant is present in an amount ranging from 0.01 wt. % to 15 wt. %, relative to the total weight of the colorant composition.

10: The colorant composition according to claim 1, wherein the liquid polar fatty substance is present in an amount ranging from 10 wt. % to 50 wt. %, relative to the total weight of the colorant composition.

11: The colorant composition according to claim 1, wherein the composition further comprises water, in an amount ranging from 15 wt. % to 80 wt. %, relative to the total weight of the colorant composition.

12: A dye kit, comprising
(a) a colorant composition according to claim 1, and
(b) a developer composition, comprising at least one oxidizing agent.

13: The dye kit according to claim 12, wherein the developer composition further comprises at least one nonionic surfactant chosen from fatty alcohol-based compounds, fatty amide-based compounds, and mixtures thereof; and is present in an amount ranging from 0.1 wt. % to 20 wt. %, relative to the total weight of the developer composition.

14: The dye kit according to claim 12, wherein the oxidizing agent is chosen from hydrogen peroxide and/or one or more hydrogen peroxide-generating systems; and is present in an amount ranging from 0.1 wt. % to 25 wt. %, relative to the total weight of the developer composition.

15: A process for dyeing keratin fibers, using the dye kit according to claim 12, comprising
mixing the colorant composition and developer composition immediately before use,
applying the resulted mixture onto the keratin fibers,
rinsing off the mixture on the keratin fibers with water, and
optionally, washing the keratin fibers with a shampoo, followed by rinsing the keratin fibers with water.
